(19) United States
(12) Patent Application Publication
(10) Pub. No.: US 2016/0354422 A1
(43) Pub. Date: Dec. 8, 2016

(54) FOCUS ENHANCING COMPOSITION

(71) Applicants: Jeffrey Edward Moss, Norwalk, CT (US); Ian Edward O'Connell, Westport, CT (US); Parker Lindsley Kligerman, Westport, CT (US)

(72) Inventors: Jeffrey Edward Moss, Norwalk, CT (US); Ian Edward O'Connell, Westport, CT (US); Parker Lindsley Kligerman, Westport, CT (US)

(21) Appl. No.: 15/172,890

(22) Filed: Jun. 3, 2016

Related U.S. Application Data
(60) Provisional application No. 62/170,351, filed on Jun. 3, 2015.

(51) Int. Cl. A61K 36/82; A61K 31/221; A61K 31/685; A61K 47/12; A61K 31/522; A61K 47/26; A61K 31/4415; A61K 31/519; A61K 31/714; A61K 31/4188; A61K 31/197; A61K 36/258; A61K 31/198; A61K 31/455

(52) U.S. Cl. CPC A61K 36/82; A61K 31/198; A61K 31/221; A61K 31/685; A61K 47/12; A61K 31/522; A61K 31/455; A61K 31/4415; A61K 31/519; A61K 31/714; A61K 31/4188; A61K 31/197; A61K 36/258; A61K 47/26

(57) ABSTRACT

A focus enhancing composition is provided. The focus enhancing composition includes a plurality of members. The plurality of members at least include L-Theanine, Acetyl L-Canitine and a choline derivative. The plurality of members are present in an amount effective to raise memory and cognition levels when administered to a mammal.

### CROSS-REFERENCE TO RELATED APPLICATION

This application claims the benefit of priority of U.S. provisional application No. 62/170,351, filed Jun. 3, 2015, the contents of which are herein incorporated by reference.

### BACKGROUND OF THE INVENTION

The present invention relates to a composition and, more particularly, to a focus enhancing composition.

Currently, a method of enhancing one's ability to focus is to ingest pills, powders or drinks that are generally not palatable. Many of these and other existing compositions fail to provide a suitable enhanced focus, and consumers are constantly looking for improving products. Most medications for enhancing focus are regulated, potentially harmful and addictive, and are unavailable to the general public.

As can be seen, there is a need for an improved focus enhancing composition.

### SUMMARY OF THE INVENTION

In one aspect of the present invention, a focus enhancing composition comprises a plurality of members comprising L-Theanine, Acetyl L-Canitine and a choline derivative, wherein the plurality of members are present in an amount effective to raise memory and cognition levels when administered to a mammal.

These and other features, aspects and advantages of the present invention will become better understood with reference to the following drawings, description and claims.

### BRIEF DESCRIPTION OF THE DRAWINGS

FIG. 1 is a schematic view illustrating the ingredients of an embodiment of the present invention; and

FIG. 2 is a schematic view illustrating additional ingredients of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes an edible composition that provides an individual with enhanced focus when consumed. The present invention provides an edible composition intended to enhance an individual's ability to focus and stay on task. In certain embodiment, the edible composition includes at least one flavorant in a sufficient amount to render the composition palatable. In certain embodiments, the composition is mixed with water, making it easy to ingest. The present invention makes it easy, safe and enjoyable for individuals to enhance their focus. The present invention is palatable, convenient and effective for enhancing an individual's ability to focus.

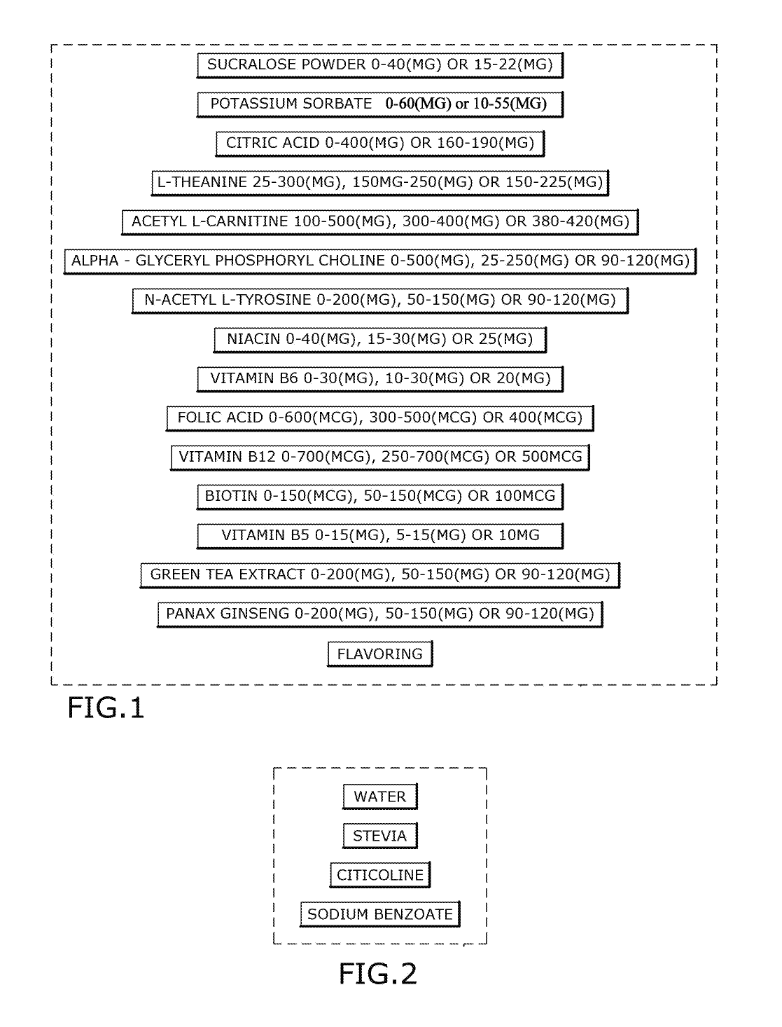

Referring to FIGS. 1 and 2, the composition of the present invention may include a composition having a plurality of members. The plurality of members may include L-Theanine, Acetyl L-Carnitine, and a choline derivative. The focus-enhancing composition may also include caffeine, vitamins, nutrients, amino acids, preservatives, water and the like. The plurality of members are present in an amount effective to raise memory and cognition levels when administered to a mammal, such as a human.

The benefits of the ingredients include the following. Alpha Glyceryl Phosphoryl Choline delivers Choline to the brain, aiding in memory and cognition. Acetyl L-Carnitine is broken down by blood into Carnitine which is used by the body to transport fatty acids to the mitochondria for breakdown, in a process that can improve memory and focus while preventing brain cell deterioration. L-Theanine increases levels of serotonin, dopamine, GABA and glycine levels in the brain, producing calming effects, improving cognition and exhibiting antioxidant effects.

The present invention may further include the following ingredients: Sucralose Powder, Potassium Sorbate, Citric Acid, Caffeine, N-Acetyl L-Tyrosine, Niacin, Vitamin B6, Folic Acid, Vitamin B12, Biotin, Vitamin B5, Green Tea Extract, and Panax Ginseng.

The benefits of these ingredients include the following. Panax Ginseng improves mental functions such as concentration and memory. Caffeine stimulates the Central Nervous System, producing a number of possible effects, such as alertness and energy. Combining Caffeine with L-Theanine can enhance ones focus and mental functions. N-Acetyl-L-Tyrosine is converted by the body into L-Dopa, which is used to make Dopamine. This can produce benefits on numerous brain functions, notably increasing energy levels and concentration. Green Tea Extract exhibits antioxidant effects on the brain, therefore protecting one's memory. Niacin (Vitamin B3) is good for one's health, and can help regulate cholesterol levels and heart health. Vitamin B6 (Pyridoxine) aids in the synthesis of numerous neurotransmitters, therefore aiding in many mental functions. Folate (Folic Acid/Vitamin B9) is used by the body to repair DNA and is essential for numerous other bodily functions. Vitamin B12 (cobalamin) aids in the metabolism of amino acids. Biotin (Vitamin B7) is utilized in the metabolism of fats and amino acids. Pantothenic Acid (Vitamin B5) is an essential nutrient, and synthesizes coenzyme-A as well as synthesizes and metabolizes many fats, proteins and carbohydrates.

The above mentioned supplements work together to protect the brain and to aid the brain in a number of functions, most significantly to enhance one's ability to focus. A flavorant is used to alter the taste of the above mentioned ingredients. Sucralose or a different sweetener is used to alter the taste, and combined with at least one flavorant, together help make the edible composition palatable to consumers. Citric Acid is used as a preservative. Potassium sorbate is used as a preservative. Water can be used to dissolve ingredients, and make ingesting the invention easier and more pleasant. All of these ingredients work together as a whole to create a palatable edible composition that can enhance one's ability to focus.

The present invention may include the following ingredients with the exemplary composition proportions: Sucralose Powder about 0-40 (mg) or about 15-22 (mg); Potassium Sorbate about 0-60 (mg) or about 10-55 (mg); Citric Acid about 0-400 (mg) or about 160-190 (mg); L-Theanine about 25-300 (mg), about 150 mg-250 (mg) or about 150-225 (mg); Acetyl L-Carnitine about 100-500 (mg), about 300-400 (mg) or about 380-420 (mg); Caffeine about 0-200 (mg), about 75-200 (mg) or about 90-120 (mg); Alpha-Glyceryl Phosphoryl Choline about 0-500 (mg), about 25-250 (mg) or about 90-120 (mg); N-Acetyl L-Tyrosine about 0-200 (mg), about 50-150 (mg) or about 90-120 (mg); Niacin about 0-40 (mg), about 15-30 (mg) or about 25 (mg); Vitamin B6 about 0-30 (mg), about 10-30 (mg) or about 20 (mg); Folic Acid about 0-600 (mcg), about 300-500 (mcg) or about 400 (mcg); Vitamin B12 about 0-700 (mcg), about 250-700 (mcg) or about 500 mcg; Biotin about 0-150 (mcg), about 50-150 (mcg) or about 100 mcg; Vitamin B5 about 0-15 (mg), about 5-15 (mg) or about 10 (mg); Green Tea Extract about 0-200 (mg), about 50-150 (mg) or about 90-120 (mg); and Panax Ginseng about 0-200 (mg), about 50-150 (mg) or about 90-120 (mg).

A method of making the composition may include the following. Add water to the mixing tank withholding at least 5% of total water to adjust final blend if necessary and begin stirring. Water may be constantly stirred for the ingredients to mix into the solution. Add Sucralose (powder), Potassium Sorbate and blend for 10 minutes. Another sweetener may be used in this step such as Stevia. Additional preservatives may be added as well such as Sodium Benzoate or removed entirely. Add L-Theanine, Acetyl L-Carnitine, Caffeine and blend for 5 minutes. Add Alpha-Glyceryl Phosphoryl Choline, N-Acetyl L-Tyrosine, Niacin, Vitamin B6, Folic Acid, Vitamin B12, Biotin, Vitamin B5 and blend for 5 minutes. Another choline source may be used for this step, such as Citicholine. Add Green Tea Extract, Panax Ginseng, Berry Flavor and blend for 5 minutes. Add Citric Acid and blend for 15-30 minutes. Blending times are approximate depending on batch size and blending equipment. Adjust times to compensate for these variables.

A person orally ingests the present invention in order to feel desired effects. The vitamins, amino acids and antioxidants enable users to feel calm, focused and alert without the jitteriness associated with other caffeinated products. Users consume the product whenever they feel they need a mental boost and sustained concentration such as in an athletic event, work or school. For example, a person may orally ingest about 1 up to about 5 fluid ounces, such as about 2 fluid ounces per serving. Effects typically last between 4-6 hours.

It should be understood, of course, that the foregoing relates to exemplary embodiments of the invention and that modifications may be made without departing from the spirit and scope of the invention as set forth in the following claims.

What is claimed is:

1. A focus enhancing composition comprising a plurality of members comprising L-Theanine, Acetyl L-Canitine and a choline derivative, wherein the plurality of members are present in an amount effective to raise memory and cognition levels when administered to a mammal.

2. The composition of claim 1, wherein the choline derivative comprises alpha-glyceryl phosphoryl choline.

3. The composition of claim 2, comprising the plurality of members in the proportions of:
about 25-200 mg L-Theanine,
about 100-500 mg Acetyl L-Canitine, and
about 5 mg-500 mg choline derivative.

4. The composition of claim 1, wherein the plurality of members further comprises at least one or more of Sucralose Powder, Potassium Sorbate, Citric Acid, Caffeine, N-Acetyl L-Tyrosine, Niacin, Vitamin B6, Folic Acid, Vitamin B12, Biotin, Vitamin B5, Green Tea Extract, and Panax Ginseng.

5. The composition of claim 1, wherein the plurality of members further comprise Sucralose Powder, Potassium Sorbate, Citric Acid, Caffeine, N-Acetyl L-Tyrosine, Niacin, Vitamin B6, Folic Acid, Vitamin B12, Biotin, Vitamin B5, Green Tea Extract, and Panax Ginseng.

6. The composition of claim 4, comprising the plurality of members in the proportions of:
about 0-40 mg Sucralose Powder,
about 0-60 mg Potassium Sorbate,
about 0-400 mg Citric Acid,
about 25-300 mg L-Theanine,
about 100-500 mg Acetyl L-Carnitine,
about 0-200 mg Caffeine,
about 5-500 mg Alpha-Glyceryl Phosphoryl Choline,
about 0-200 mg N-Acetyl L-Tyrosine,
about 0-40 mg Niacin,
about 0-30 mg Vitamin B6,
about 0-600 mcg Folic Acid,
about 0-700 mcg Vitamin B12,
about 0-150 mcg Biotin,
about 0-15 mg Vitamin B5,
about 0-200 mg Green Tea Extract, and
about 0-200 mg Panax Ginseng.

7. The composition of claim 4, comprising the plurality of members in the proportions of:
about 15-22 mg Sucralose Powder,
about 10-55 mg Potassium Sorbate,
about 160-190 mg Citric Acid,
about 150-225 mg L-Theanine,
about 380-420 mg Acetyl L-Carnitine,
about 90-120 mg Caffeine,
about 90-120 mg Alpha-Glyceryl Phosphoryl Choline,
about 90-120 mg N-Acetyl L-Tyrosine,
about 15-30 mg Niacin,
about 10-30 mg Vitamin B6,
about 300-500 mcg Folic Acid,
about 250-700 mcg Vitamin B12,
about 50-150 mcg Biotin,
about 5-15 mg Vitamin B5,
about 90-120 mg Green Tea Extract, and
about 90-120 mg Panax Ginseng.

8. The composition of claim 1, wherein the plurality of members further comprises at least one of flavoring, water, stevia, citicoline, and sodium benzoate.

9. The composition of claim 1, wherein the plurality of members further comprises flavoring, water, stevia, citicoline, and sodium benzoate.

10. A method of elevating memory and cognition levels in a mammal in need thereof comprising the oral administration thereto of a composition according to claim 1.
